(19) United States
(12) Patent Application Publication
(10) Pub. No.: US 2014/0011229 A1
(43) Pub. Date: Jan. 9, 2014

(54) FILTER SEPARATION METHOD AND FILTER SEPARATION APPARATUS USED FOR THE SAME

(71) Applicant: Hitachi, Ltd., Tokyo (JP)

(72) Inventors: Yuta NAKATSUKA, Tokyo (JP); Shuichi MORI, Tokyo (JP); Koji SEO, Osaka (JP); Noe MIYASHITA, Matsudo-shi (JP)

(21) Appl. No.: 13/930,041

(22) Filed: Jun. 28, 2013

(30) Foreign Application Priority Data
Jul. 3, 2012 (JP) 2012-149251

(51) Int. Cl. C12Q 1/24; C12M 1/00

(52) U.S. Cl. CPC C12Q 1/24; C12M 29/04
USPC 435/30; 435/309.1

(57) ABSTRACT

In a microorganism capturing device as a filter separation apparatus, a first contact chamber and a second contact chamber in each of which an object to be treated is contacted with a treatment agent are disposed apart from each other by a hydrophilic filter. The second contact chamber and a filtrate outlet are separated from each other by a hydrophobic filter. The microorganism capturing device is adapted to efficiently contact the object to be treated with the treatment agent, even if the object to be treated enters into the hydrophilic filter.

### CROSS REFERENCE TO RELATED APPLICATIONS

This application claims the benefit of Japanese Patent Application No. 2012-149251 filed on Jul. 3, 2012, the disclosure of which is incorporated herein by reference.

### BACKGROUND OF THE INVENTION

1. Field of the Invention

The present invention relates to a filter separation method and a filter separation apparatus used for the same.

2. Description of the Related Art

One of well-known methods of counting microorganisms is ATP method in which adenosine triphosphates (to be simply referred to as ATPs hereinafter) extracted from microorganisms are quantified and the microorganisms are thereby indirectly counted (see, for example, Japanese Laid-Open Patent Application, Publication No. H11-137293). The ATP method extracts ATPs contained in the microorganisms by bringing the captured microorganisms into contact with an ATP extracting reagent, and counts the microorganisms based on luminescence intensity obtained by making the extracted ATPs react with a luminescence reagent.

In a method of counting captured microorganisms based on, for example, the number of microorganism colonies cultured in a plate medium, it takes several days to obtain a result of the counting. On the other hand, the ATP method as described above can dramatically reduce the required time as short as only about one to several hours from when microorganisms are captured until when the microorganisms are counted.

Another counting system has been known recently in which a prescribed collecting implement collects airborne microorganisms and a measurement apparatus equipped with the collecting implement automatically counts the collected microorganisms in conformity with the ATP method (see, for example, Japanese Laid-Open Patent Application, Publication No. 2012-053057).

The collecting implement used for the counting system includes, for example: a gel-like carrier that collects microorganisms using an air sampler or the like; a housing that houses the carrier and defines a funnel part therein; and a filter that is disposed at a discharge opening formed in a lower portion of the funnel part. The filter is constituted by two filters with one on top of the other, that is, a hydrophilic filter and a hydrophobic filter in this order from a side nearer to the funnel part (from above to below).

In the counting system, plural kinds of reagents or the like are put in the funnel part of the collecting implement in a prescribed order, which allows ATPs of the microorganisms collected in the carrier to be extracted.

More specifically, each of a buffer solution (a wash solution), an ATP eliminating reagent (which eliminates an ATP present outside a cell of a microorganism), and an ATP extraction reagent, for example, is put in the funnel part according to steps in conformity with the ATP method.

Each of the reagents or the like put in the funnel part in respective steps is retained in the funnel part, without flowing out of the above-described discharge opening due to water repellency of the above-described hydrophobic filter. Out of the plural reagents or the like, a first reagent or the like is retained in the funnel part, comes in contact with the microorganisms for a prescribed time period, and is discharged by means of suction (for example, vacuuming) via the discharge opening. Similarly, a subsequent reagent or the like is retained in the funnel part, comes in contact with the microorganisms for a prescribed time period, and is discharged. That is, in the funnel part, input of the reagents or the like, contact between the reagents or the like and the microorganisms, and discharge of the reagents or the like are repeatedly conducted according to the steps in conformity with the ATP method.

After an ATP extract of the microorganisms is finally obtained in the funnel part, a prescribed amount of the ATP extract is dispensed to a luminescence-test tube of a luminescence-intensity measurement unit and is used for measuring luminescence intensity as described above.

In the above-described collecting implement (see, for example, Japanese Laid-Open Patent Application, Publication No. 2012-053057), for example, when the first reagent or the like is brought in contact with an object to be treated (a carrier in which microorganisms are captured, or the microorganisms themselves, or the like) in the funnel part and is discharged through the hydrophilic filter and the hydrophobic filter, part of the object to be treated may infiltrate in the hydrophilic filter (in fine pores of the hydrophilic filter)

In the above-described ATP method, (see, for example, Japanese Laid-Open Patent Application, Publication No. H11-137293), microorganisms are counted based on weak luminescence intensity of ATPs contained in the microorganisms. A reagent or the like is required to sufficiently contact with (or pretreat) the microorganisms. That is, there is a need for a more efficient contact of the microorganisms with the reagent or the like as a treatment agent even if the object to be treated penetrates into a hydrophilic filter, so as to count the microorganisms with high sensitivity even in the conventional counting system using the collecting implement (see, for example, Japanese Laid-Open Patent Application, Publication No. 2012-053057).

In light of the above-described problems, the present invention has been made in an attempt to provide: a filter separation method in which, when an object to be treated is separated by a filter and the separated object is treated with a treatment agent, the object to be treated can be brought in contact with the treatment agent more efficiently than in the conventional technology; and a filter separation apparatus used for the filter separation method.

### SUMMARY OF THE INVENTION

A filter separation method for filter-separating an object to be treated, in which: the object to be treated is contacted with a liquid treatment agent; a first filter and a second filter are used; and the second filter exhibits liquid repellency to the liquid treatment agent. The filter separation method includes the steps of: disposing the first filter and the second filter spaced apart from each other; and putting a mixture of the object to be treated and the treatment agent onto the first filter from a side opposite to the second filter, separating the object to be treated on the first filter, allowing the treatment agent in the mixture to pass through the first filter and to be retained between the first filter and the second filter due to the liquid repellency of the second filter, without passing through the second filter.

In the filter separation apparatus of the present invention used for the filter separation method as described above, a first contact chamber and a second contact chamber in each of which an object to be treated is brought into contact with a treatment agent are disposed apart from each other by a hydrophilic filter; and the second contact chamber and a filtrate outlet are disposed apart from each other by a hydrophobic filter.

With the present invention, the filter separation method and the filter separation apparatus for using the same are provided in which, when an object to be treated having been separated by a filter is treated with a treatment agent, the object to be treated can be brought into contact with the treatment agent more efficiently than a conventional technology.

### BRIEF DESCRIPTION OF THE DRAWINGS

FIG. 1 is a perspective diagram illustrating a microorganism capturing device as a filter separation apparatus according to an embodiment of the present invention.

FIG. 2A and FIG. 2B are each an exploded perspective diagram illustrating the microorganism capturing device (filter separation apparatus) of FIG. 1 according to the embodiment. FIG. 2A is the exploded perspective diagram as viewed downward from obliquely above. FIG. 2B is the exploded perspective diagram as viewed upward from obliquely below.

FIG. 3 is a perspective diagram for explaining a method of capturing microorganisms using the microorganism capturing device (filter separation apparatus) according to the embodiment.

FIG. 4 is a cross sectional diagram illustrating a state where the microorganism capturing device (filter separation apparatus) of FIG. 1 is mounted on a microorganism counting apparatus according to the embodiment.

FIG. 5A1 to FIG. 5A4 and FIG. 5B1 to FIG. 5B4 are each a diagram for explaining a step of a filter separation method carried out by using the microorganism capturing device (filter separation apparatus) of FIG. 1, according to the embodiment.

FIG. 5A1 to FIG. 5A4 are each a cross-sectional diagram of the microorganism capturing device for illustrating the filter separation method. FIG. 5B1 to FIG. 5B4 are each a schematic diagram illustrating a state in vicinity of a hydrophilic filter in the steps corresponding to those of FIG. 5A1 to FIG. 5A4, respectively.

FIG. 6A is a schematic diagram illustrating a state in which, in the microorganism capturing device (filter separation apparatus) of FIG. 1, microorganisms separated by a hydrophilic filter are brought in contact with an ATP extraction reagent according to the embodiment.

FIG. 6B is a schematic diagram illustrating a state in which, in a microorganism capturing device (filter separation apparatus) according to a conventional technology, microorganisms separated by a hydrophilic filter are brought in contact with an ATP extraction reagent according to the embodiment.

FIG. 7A is a partially enlarged cross sectional diagram illustrating a filter fitting portion and a vicinity thereof of a microorganism capturing device (filter separation apparatus) according to a variation of the present invention.

FIG. 7B is a perspective diagram illustrating a filter hydrophilic filter forming body and a hydrophobic filter forming body used in the microorganism capturing device of FIG. 7A with a partial cutaway of a support member of the filters, according to the variation of the present invention.

### DETAILED DESCRIPTION OF THE EXEMPLARY EMBODIMENT

Next is described an embodiment of the present invention. In a filter separation method of the present invention and a filter separation apparatus used for the filter separation method, a hydrophilic filter that separates microorganism (an object to be treated) and a hydrophobic filter that exhibits liquid repellency to a liquid reagent or the like (a treatment agent) which is brought in contact with the microorganism are disposed spaced apart from each other. Note that the hydrophilic filter may also be referred to as a “first filter”, and the hydrophobic filter, as a “second filter”. The liquid reagent or the like as the treatment agent used in the embodiment is a so-called aqueous liquid. The liquid repellency used in the embodiment means water repellency.

Below are described the microorganism capturing device as the filter separation apparatus according to the embodiment, and then, the filter separation method according to the embodiment which is carried out using the microorganism capturing device (filter separation apparatus).

### <Microorganism Capturing Device>

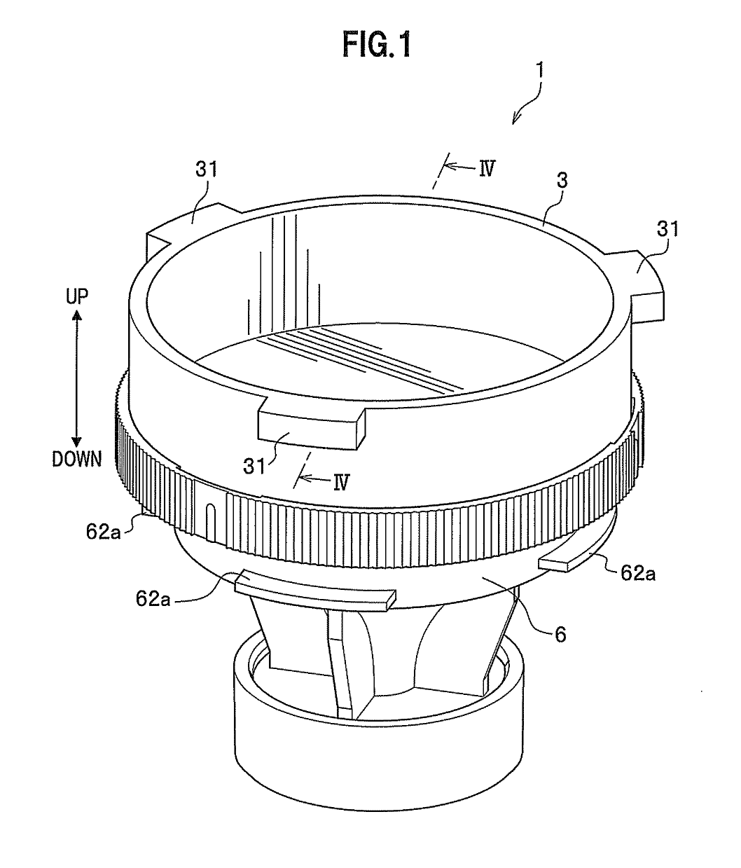

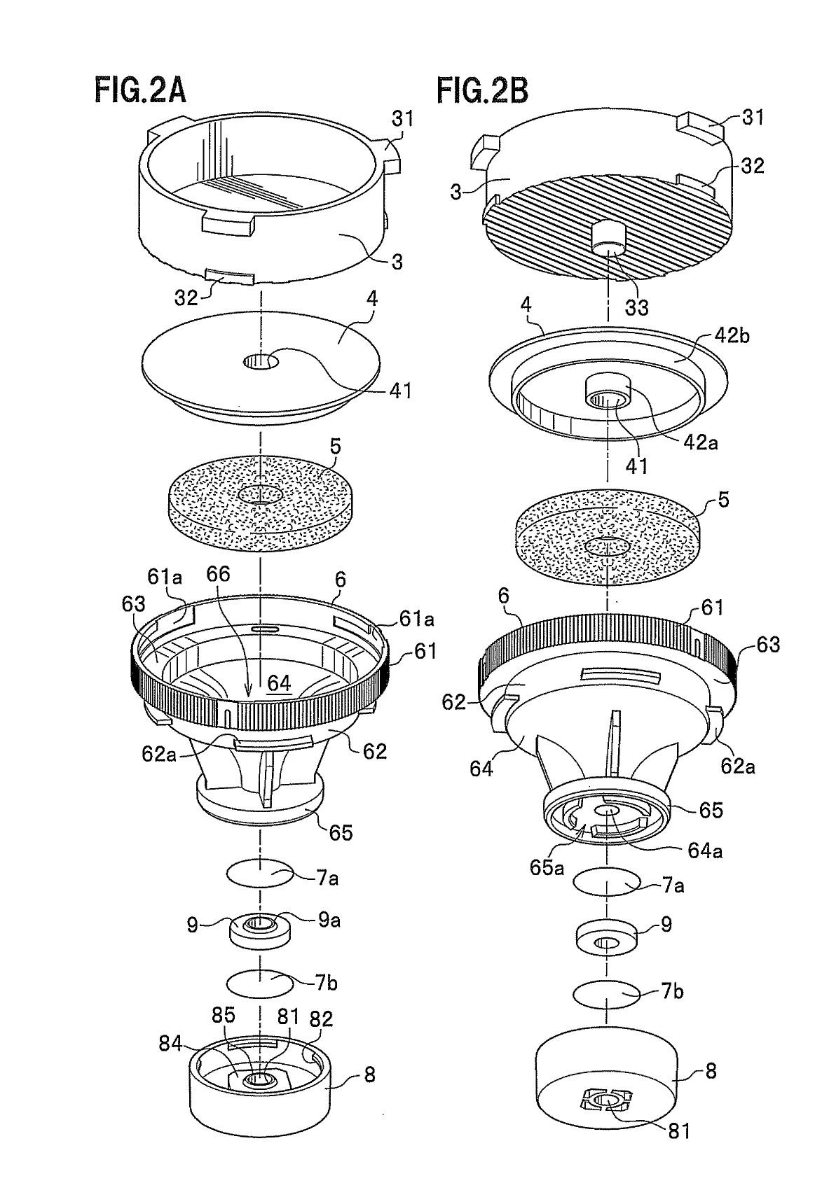

FIG. 1 is a perspective diagram illustrating the microorganism capturing device as the filter separation apparatus according to this embodiment. FIG. 2A and FIG. 2B are each an exploded perspective diagram illustrating the microorganism capturing device (filter separation apparatus) of FIG. 1. FIG. 2A is the exploded perspective diagram as viewed downward from obliquely above. FIG. 2B is the exploded perspective diagram as viewed upward from obliquely below.

Note that the microorganism capturing device 1 according to this embodiment is used for capturing so-called air-borne microorganisms (for example, microbes and fungi), details of which will be described hereinafter, and is also used for counting the captured microorganisms.

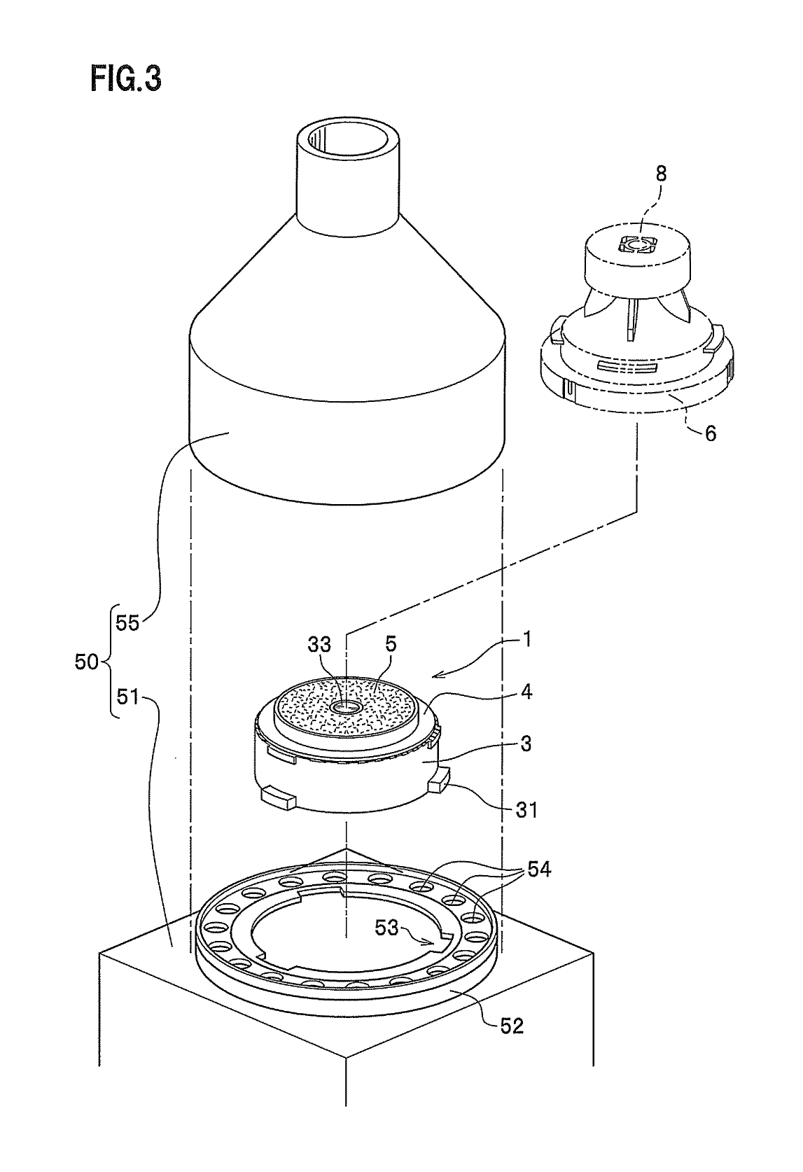

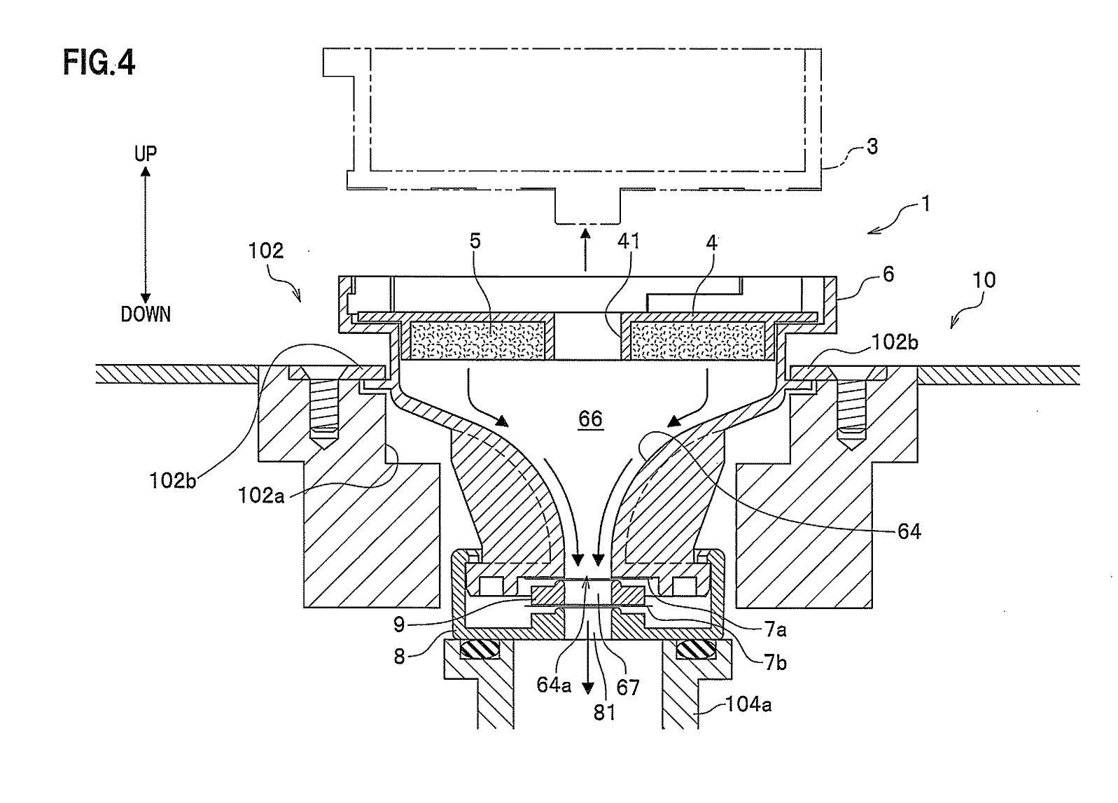

In capturing air-borne microorganisms, the microorganism capturing device 1 according to this embodiment is placed in an impactor-type air sampler 50 to be described hereinafter (see FIG. 3). In counting the captured microorganisms, the microorganism capturing device 1 is, in turn, mounted in the microorganism counting apparatus 10 to be described hereinafter (see FIG. 4). Though described in detail later, the microorganism capturing device 1 is used upside down in capturing and counting microorganisms as described in detail hereinafter (see FIG. 3 and FIG. 4).

As shown in FIG. 1, the microorganism capturing device 1 has an upper portion thereof formed in a substantially cylindrical shape and a lower portion thereof formed in an inverted conical shape such that the lower a horizontal cross section of the microorganism capturing device 1, the smaller a diameter of the horizontal cross section. As described in detail below, the microorganism capturing device 1 has the upper portion which is engaged with the air sampler 50 (see FIG. 3) and is used for capturing microorganisms, and also has the lower portion which is engaged with the microorganism counting apparatus 10 (see FIG. 4) and is used for counting the captured microorganisms.

In FIG. 1, reference numeral 3 indicates a cover; reference numeral 6, a housing; reference numeral 31, second engaging claws 31 engaging with the air sampler 50 (see FIG. 3) to be described hereinafter; and reference numeral 62a, first engagement claws engaging with the microorganism counting apparatus 10 (see FIG. 4).

The microorganism capturing device 1 (see FIG. 1) includes, as shown in FIG. 2A and FIG. 2B, the cover 3, a capturing dish 4, a carrier 5, the housing 6, a hydrophilic filter 7a, a spacer 9, a hydrophobic filter 7b, and a filter holding cap 8, which are mounted in this order from above to below.

The cover 3 is disposed so as to close an upper opening of the housing 6 to be described hereinafter and has a cylindrical shape with a bottom and an opening facing upward. On an upper circumferential edge of the outer circumferential surface of the cover 3, the second engaging claws 31 described above are formed to protrude radially outward and are disposed in constant spacing along a circumferential direction of the cover 3. In this embodiment, the number of the second engaging claws 31 is three in accordance with the number of engaging cutout portions 53 (see FIG. 3) of the air sampler 50 (see FIG. 3) to be described hereinafter.

On a lower circumferential edge of the circumferential surface of the cover 3, three third engaging claws 32 are formed to protrude radially outward and are disposed in constant spacing along the circumferential direction of the cover 3. The third engaging claws 32 are fitted in respective first L-shaped grooves 61a to be described hereinafter of the housing 6, to thereby detachably engage the cover 3 with the housing 6.

As shown in FIG. 2B, an outer bottom surface of the cover 3 is formed by an uneven surface made up of multiple straight ridges protruding downward and disposed in parallel with each other. When the outer bottom surface is disposed to come in contact with an upper surface of the capturing dish 4 to be described hereinafter, the uneven outer bottom surface can reduce an area of contact with the capturing dish 4. For example, when the microorganism capturing device 1 is disposed at a mounting unit 102 (see FIG. 4) to be described hereinafter of the microorganism counting apparatus 10 (see FIG. 4) and the cover 3 is then detached from the housing 6, the uneven surface allows the cover 3 to be easily detached from the capturing dish 4, leaving the capturing dish 4 on the housing 6. In another case, microorganisms are captured using the air sampler 50 (see FIG. 3) and are kept cold according to necessity when transported to an appropriate facility for counting the microorganisms (for example, a facility equipped with the microorganism counting apparatus 10 (see FIG. 4)), which is to be described hereinafter. In this case, condensation may occur, though not frequently, between the cover 3 and the capturing dish 4. Even so, the uneven surface facilitates an easy detachment of the cover 3 from the capturing dish 4. Such an uneven surface is not limited to be made up of the above-described straight ridges and may be made up of, for example, a plurality of protrusions or grains of a satin finished pattern, a weave pattern, and the like.

As shown in FIG. 2B, a cylindrically-shaped downwardly-protruding protrusion 33 is formed on the outer bottom surface of the cover 3. The protrusion 33 has an outer diameter slightly smaller than an inner diameter of a through hole 41 to be described next of the capturing dish 4. A height of the protrusion 33 is equal to a depth of the through hole 41.

As shown in FIG. 2A and FIG. 2B, the capturing dish 4 is disk-shaped. The capturing dish 4 has the through hole 41 which penetrates through a central portion between the upper surface and a bottom surface of the capturing dish 4.

As shown in FIG. 2A, the upper surface of the capturing dish 4 forms an even surface so as to allow contact with the outer bottom surface of the cover 3 described above.

Inner and outer ring-shaped ribs 42a, 42b are vertically formed on the lower surface of the capturing dish 4 so as to accommodate therebetween a ring-shaped carrier 5 to be described later.

The outer diameter of the capturing dish 4 is set to be within a range not smaller than an inner diameter of a lower cylinder portion 62 of the housing 6 and not larger than an inner diameter of an upper cylinder portion 61 of the housing 6 to be described hereinafter. The outer diameter of the capturing dish 4 is preferably but not necessarily set to be substantially equal to the inner diameter of the upper cylinder portion 61. An outer diameter of the outer ring-shaped rib 42b of the capturing dish 4 shown in FIG. 2B is set to be not larger than the inner diameter of the lower cylinder portion 62 and is preferably but not necessarily set to be substantially equal to the inner diameter of the lower cylinder portion 62.

The carrier 5 is placed in the air sampler 50 (see FIG. 3) as to be described hereinafter, receives an air flow when the air sampler 50 sucks air, and captures microorganisms accompanied by the air flow.

The carrier 5 is made of a material which undergoes a phase transition from gel to sol when a temperature of the carrier 5 is raised from room temperature. The carrier 5 is preferably made of such a material that undergoes a phase transition from gel to sol at 30 degrees C. or higher. More preferably, the material liquefies at a temperature from 37 degrees C. to 40 degrees C. Most preferably, the material is a gelatin, a mixture of gelatin and glycerol, or a copolymer having a ratio of N-acryloylglycinamide to N-methacryloyl-N′-biotinyl propylene diamine of 10:1.

The carrier 5 is ring-shaped as described above and is preferably but not necessarily has a shape same as a space defined between the ring-shaped ribs 42a, 42b of the capturing dish 4 as shown in FIG. 2B.

The carrier 5 is formed by applying the above-described material to the space between the ring-shaped ribs 42a, 42b, or filling the space with the material. Or, the carrier 5 having a self-standing ring shape is disposed by fitting into the space.

As shown in FIGS. 2A and 2B, the housing 6 has: the upper cylinder portion 61 having the inner diameter substantially the same as the outer diameter of the cover 3 as described above; the lower cylinder portion 62 having the inner diameter smaller than the inner diameter of the upper cylinder portion 61; a conical portion 64 formed in an inverted conical shape with an inner diameter which gradually becomes smaller from the inner diameter of the lower cylinder portion 62 downward; and a filter fitting portion 65 provided around an outlet of the discharge opening 64a formed in the lowest portion of the conical portion 64, which are disposed in this order to form an integral unit.

A first contact chamber 66 is defined in a space inside the conical portion 64. Microorganisms captured by the carrier 5 are brought into contact with a reagent or the like in the first contact chamber 66, details of which will be described later.

On an inner circumferential surface of the upper cylinder portion 61, three first L-shaped grooves 61a into which respective third engaging claws 32 of the cover 3 are fitted are formed along the inner circumferential surface at positions corresponding to the respective third engaging claws 32 as described above.

The lower cylinder portion 62 is coupled with the upper cylinder portion 61 via a shelf portion 63.

On an outer circumferential surface of the lower cylinder portion 62, the first engagement claws 62a are formed each of which is engaged with the engaging ring 102b (see FIG. 4) of the microorganism counting apparatus 10 (see FIG. 4) to be described hereinafter. The first engagement claws 62a protrude outwardly in a radial direction of the lower cylinder portion 62, and are disposed in constant spacing along a circumferential direction thereof. In this embodiment, for example, four first engagement claws 62a are formed.

The conical portion 64 has an inner diameter which gradually becomes smaller downward and ends up with a discharge opening 64a (see FIG. 2B) at a bottom thereof.

Though details will be described later (see FIG. 4), the conical portion 64 has an inner wall surface which is smoothly curved with undulations from a top thereof down to the discharge opening 64a. This allows contents put in the conical portion 64 to easily flow down to the discharge opening 64a.

The filter fitting portion 65 has a thin disk-like shape. The filter fitting portion 65 has, at a central portion thereof, the discharge opening 64a (see FIG. 2B) so as to communicate with the first contact chamber 66. A space surrounding an outlet of the discharge opening 64a is defined as a filter housing portion 65a for the hydrophilic filter 7a. The hydrophilic filter 7a disposed in the filter housing portion 65a is designed to close the discharge opening 64a.

The hydrophilic filter 7a is made of material such as, but not limited to, cellulose acetate, cellulose nitrate, polysulfone, polycarbonate, and polyester.

The hydrophilic filter 7a may be selected from commercially available products such as, for example, MF-Millipore (manufactured by Nihon Millipore K.K.), Durapore (Nihon Millipore K.K.), Isopore (Nihon Millipore K.K.), Cyclopore (Whatman Japan K.K.) JCWP01300 (Millipore K.K.), Millex LC (Nihon Millipore K.K.), K040A013A (Advantec K.K.), and T300A013A (Advantec K.K.).

As shown in FIG. 2A and FIG. 2B, the spacer 9 is formed by a ring member. The spacer 9 is interposed between the hydrophilic filter 7a described above and the hydrophobic filter 7b to be described next. A second contact chamber 67 (see FIG. 4) is thereby defined in an inside of the spacer 9 (ring member) by being sandwiched between the hydrophilic filter 7a and the hydrophobic filter 7b.

The spacer 9 has a ring-shaped rib 9a formed on an upper surface (or a surface nearer to the hydrophilic filter 7a) around an opening in a central portion thereof. The ring-shaped rib 9a is adapted to press the hydrophilic filter 7a against the filter fitting portion 65.

The hydrophobic filter 7b is made of material such as, but not limited to, polytetrafluoroethylene, nylon, polypropylene, and glass.

The hydrophobic filter 7b may be selected from commercially available products such as, for example, gore-tex (registered trademark, manufactured by Japan Gore-Tex K.K.), MICRO-TEX (Nitto Denko K.K.), and polypropylene filter (Nihon Millipore K.K.).

The hydrophobic filter 7b may be made of the above-described hydrophilic filter 7a which is rendered hydrophobic by treating with a fluorine or silicone water repellent agent.

As shown in FIG. 2A and FIG. 2B, the filter holding cap 8 has a cylindrical shape with a thin bottom. A filtrate outlet 81 is formed in a central portion on the bottom thereof. The filter holding cap 8 is fitted onto the above-described filter fitting portion 65 from below, to thereby fix the hydrophilic filter 7a, the spacer 9, and the hydrophobic filter 7b into the filter fitting portion 65.

The filter holding cap 8 has four engagement protrusions 82 which are formed along an inner circumferential edge of a top opening portion with equal spacing along the circumferential edge. The engagement protrusions 82 are adapted to engage with a circumferential edge of the filter fitting portion 65, to thereby fix the filter holding cap 8 onto the filter fitting portion 65.

The filter holding cap 8 also has a seat portion 84 which projects from an inner bottom surface thereof toward the top opening portion, as shown in FIG. 2A. The seat portion 84 has a ring-shaped rib 85 which stands around an opening of the filtrate outlet 81 which penetrates through the seat portion 84. The ring-shaped rib 85 is adapted to press the hydrophobic filter 7b against the spacer 9.

As described above and shown in FIG. 2A and FIG. 2B, in the microorganism capturing device 1: the capturing dish 4 is mounted on the shelf portion 63 of the housing 6; and the housing 6 is coupled to the cover 3 via the capturing dish 4 using the first L-shaped grooves 61a and the second engaging claws 31. This allows the through hole 41 of the capturing dish 4 to be sealed by the protrusion 33 of the cover 3.

The housing 6 is decoupled from the cover 3 by rotating the housing 6 relative to the cover 3 so as to disengage the second engaging claws 31 from the first L-shaped grooves 61a.

The hydrophilic filter 7a is disposed in the filter housing portion 65a so as to close the discharge opening 64a of the conical portion 64. Then the spacer 9 and the hydrophobic filter 7b are disposed onto the hydrophilic filter 7a. Finally the filter holding cap 8 is fitted onto the filter housing portion 65a. This allows the hydrophilic filter 7a, the spacer 9, and the hydrophobic filter 7b to be attached to the housing 6.

The microorganism capturing device 1 as described above other than the filters 7a, 7b may be molded with resin, for example, preferably but not necessarily polypropylene.

### <Method of Capturing Microorganisms Using Microorganism Capturing Device>

Next is described a method of capturing microorganisms using the microorganism capturing device 1 (see FIG. 1) according to the embodiment. FIG. 3 to be referred to herein is a perspective diagram for explaining a method of capturing microorganisms using the microorganism capturing device (filter separation apparatus) according to the embodiment.

As shown in FIG. 3, in capturing microorganisms, the microorganism capturing device 1 is used in such a way that the capturing dish 4 for holding the carrier 5 is mounted on the cover 3. In other words, when used, the microorganism capturing device 1 shown in FIG. 1 is turned upside down and the housing 6 as well as the filter holding cap 8 are removed, while the capturing dish 4 is left on the cover 3 as it is. In removing the housing 6 from the cover 3, the cover 3 is appropriately positioned and disposed on a pedestal 52 of the air sampler 50, which will be described later. The housing 6 is then rotated relative to the cover 3 so as to disengage the second engaging claws 31 (see FIG. 2A) from the first L-shaped grooves 61a (see FIG. 2A) as described above.

The microorganism capturing device 1 is mounted on the pedestal 52 which is formed on an upper side of an air sampler body 51 of the air sampler 50 and is round-shaped as viewed from above. The pedestal 52 has, as described above, the engaging cutout portions 53 for receiving the second engaging claws 31 of the cover 3. The microorganism capturing device 1 is positioned in a central portion of the pedestal 52.

In FIG. 3, reference numeral 54 indicates suction openings of a sampler body 51. Reference numeral 55 is a nozzle head of the air sampler 50.

In the method of capturing microorganisms, as shown in FIG. 3, the microorganism capturing device 1 is mounted onto the pedestal 52. At this time, the microorganism capturing device 1 is in a state in which the carrier 5 is exposed as a result of removing the housing 6 together with the filter-securing ring 8. The nozzle head 55 is disposed over the pedestal 52.

A fan not shown disposed in the air sampler body 51 is activated so as to suck air through the suction openings 54. Air flow is then injected from a plurality of fine nozzles (not shown) provided in the nozzle head 55 to the carrier 5. As a result, microorganisms accompanied by the air injected to the carrier 5 are captured by the carrier 5. That is, in capturing microorganisms, the carrier 5 is turned upward.

As shown in FIG. 3, the protrusion 33 of the cover 3 seals the through hole 41 (see FIG. 2A and FIG. 2B) of the capturing dish 4. The protrusion 33 thus makes a surface of the capturing dish 4 nearer to the carrier 5 flush. This reduces disturbance of the received air flow. The carrier 5 can thus capture microorganisms efficiently.

After the air sampler 50 sucks a prescribed amount of air, a step of capturing microorganisms using the microorganism capturing device 1 is finished.

After completion of the capturing step, the housing 6 together with the filter holding cap 8 are fitted onto the cover 3 again. The microorganism capturing device 1 returns to its original state as shown in FIG. 1.

### <Filter Separation Method Using Microorganism Capturing Device>

Next is described a filter separation method using the microorganism capturing device 1 (filter separation apparatus) according to this embodiment. The description also includes a method of counting microorganisms captured by the capturing device 1 (see FIG. 3) according to this embodiment.

After completion of the above-described capturing step, a user conveys the microorganism capturing device 1 back to the original state shown in FIG. 1, to a facility in which the above-described microorganism counting apparatus (not shown) is installed.

The microorganism counting apparatus used herein may be any well-known apparatus adapted for counting microorganisms captured by the microorganism capturing device 1 in accordance with the ATP method. The ATP method requires supply of respective prescribed amounts of sterile hot water, functional fluid such as a buffer solution, and various reagents to the microorganism capturing device 1 (see FIG. 1) mounted on the microorganism counting apparatus, in a prescribed order. The microorganism counting apparatus used may be selected from commercially available products such as, for example, Biomaytector (registered trademark, manufactured by Hitachi Plant Technology K.K.).

FIG. 4 is a cross sectional diagram illustrating a state where the microorganism capturing device (filter separation apparatus) of FIG. 1 is mounted on the microorganism counting apparatus according to the embodiment. A cross section of the microorganism capturing device 1 shown in FIG. 4 corresponds to a cross section of that shown in FIG. 1 when cut along the line IV-IV.

As shown in FIG. 4, the microorganism counting apparatus used in this embodiment includes the mounting unit 102 having a recessed portion 102a which houses the microorganism capturing device 1 (housing 6). The recessed portion 102a is made of circularly-formed highly heat conductive material such as, for example, an aluminum member. A heater (not shown) is embedded around the recessed portion 102a.

The heater is adapted to heat the microorganism capturing device 1 disposed in the recessed portion 102a, which is to be described hereinafter.

Though details will be described later, the heater heats the carrier 5, which becomes solated, to thereby fall from the capturing dish 4 down to the conical portion 64. Meanwhile, the conical portion 64 has, as described above, the inner wall surface which is smoothly curved with undulations from the top thereof down to the discharge opening 64a. This allows the solated carrier 5 to easily flow down to the discharge opening 64a.

The microorganism capturing device 1 is disposed in the recessed portion 102a. When the cover 3 is removed from the microorganism capturing device 1, the through hole 41 of the capturing dish 4 disposed in the housing 6 becomes exposed.

In the microorganism capturing device 1, the capturing dish 4 having the through hole 41 is disposed in an upper side of the housing 6. The carrier 5 as a sample is disposed on a lower side of the capturing dish 4 as well as in the first contact chamber 66 of the housing 6.

In FIG. 4, designated at the reference numeral 64a is the discharge opening of the housing 6; at 7a, the hydrophilic filter; at 7b, the hydrophobic filter; at 8, the filter holding cap; at 9, the spacer; at 67, the second contact chamber; at 81, the filtrate outlet; at 102b, the above-described engaging ring; and at 104a, the suction head connected to a suction pump not shown.

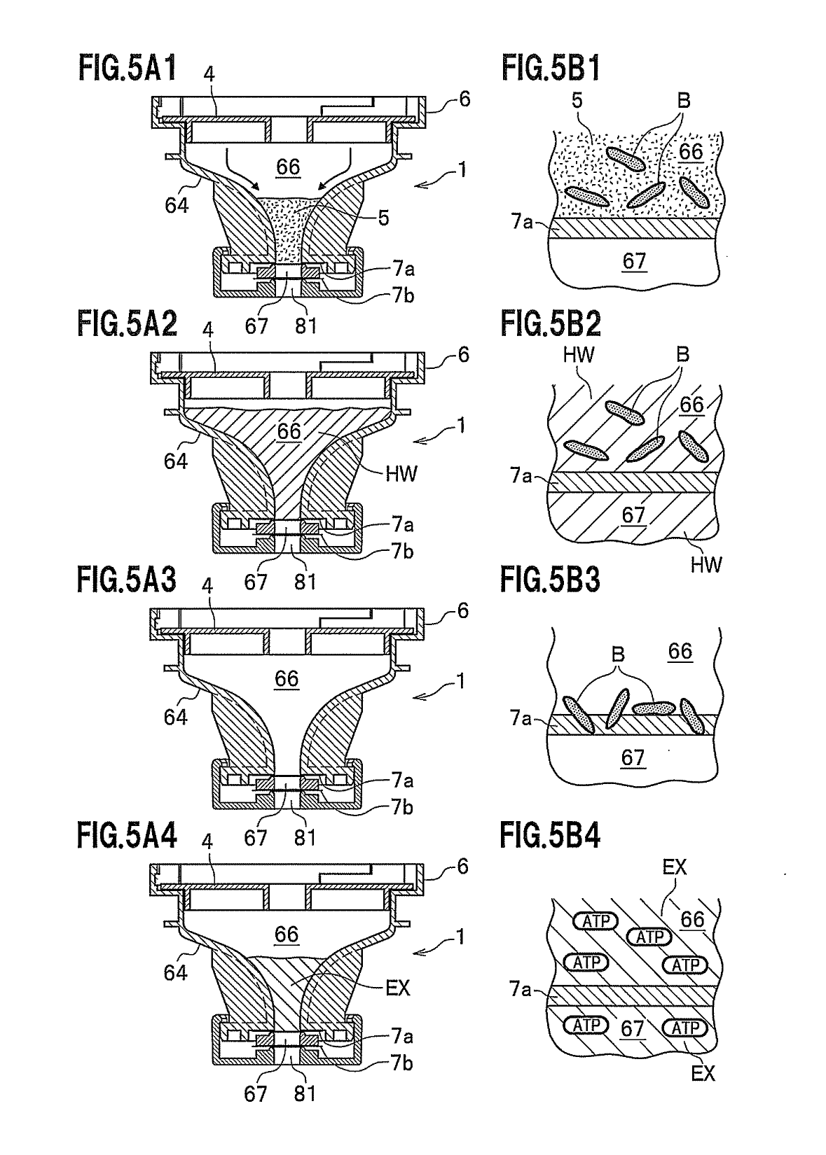

FIG. 5A1 to FIG. 5A4 and FIG. 5B1 to FIG. 5B4 referred to next are each a diagram for explaining a step of the filter separation method carried out by using the microorganism capturing device 1 (filter separation apparatus) of FIG. 1, according to the embodiment. FIG. 5A1 to FIG. 5A4 are each a cross-sectional diagram of the microorganism capturing device for illustrating the filter separation method. FIG. 5B1 to FIG. 5B4 are each a schematic diagram illustrating a state in vicinity of the hydrophilic filter 7a in the steps corresponding to those of FIG. 5A1 to FIG. 5A4, respectively.

In FIG. 5A1 to FIG. 5A4 and FIG. 5B1 to FIG. 5B4, designated at the reference numeral 1 is the microorganism capturing device; at 4, the capturing dish; at 5, the carrier; at 6, the housing; at 7a, the hydrophilic filter; at 7b, the hydrophobic filter; at 64, the conical portion; at 66, the first contact chamber; at 67, the second contact chamber; at 81, the filtrate outlet; at B, a microorganism; at EX, an ATP extraction reagent; and, at HW, the hot water.

The microorganism B shown in FIG. 5B1 to FIG. 5B4 actually has a micrometer size. An ATP shown therein actually has a molecular size. FIG. 5B1 to FIG. 5B4 are thus not illustrated based on an actual relative scale.

The above-described heater (not shown) embedded in the aluminum member which the recessed portion 102a (see FIG. 4) is made of heats the carrier 5 of the microorganism capturing device 1 (see FIG. 4). As shown in FIG. 5A1, the heated carrier 5 held in the capturing dish 4 thereby falls down to a lower portion of the first contact chamber 66 formed in the conical portion 64. In this step, as shown in FIG. 5B1, the microorganism B captured by the air sampler 50 (see FIG. 3) exists together with the carrier 5 on the hydrophilic filter 7a.

As shown in FIG. 5A2, the hot water HW is put in the first contact chamber 66, which further facilitates solation of the carrier 5 and makes the carrier 5 diluted with the hot water HW. In this step, the hydrophilic filter 7a allows the hot water HW containing a component of the carrier 5 to pass therethrough. The hydrophobic filter 7b will not allow the hot water HW to pass therethrough due to water repellency (liquid repellency) thereof. The second contact chamber 67 is thereby filled with the hot water HW. As shown in FIG. 5B2, the microorganisms B together with the hot water HW containing the component of the carrier 5 are retained in the first contact chamber 66, without passing through the hydrophilic filter 7a.

The suction head 104a (see FIG. 4) sucks the hot water HW containing the component of the carrier 5 in the first contact chamber 66, to thereby transfer the sucked hot water HW to the second contact chamber 67 via the hydrophilic filter 7a. The hot water HW containing the component of the carrier 5 in the second contact chamber 67 is then discharged out of the microorganism capturing device 1 via the hydrophobic filter 7b and the filtrate outlet 81.

As a result, as shown in FIG. 5A3, both the first contact chamber 66 and the second contact chamber 67 are filled with air, instead of the hot water HW containing the component of the carrier 5.

As shown in FIG. 5B3, the microorganisms B (see FIG. 5B1) contained in the carrier 5 are filtered out by the hydrophilic filter 5a. At this time, one or more of the microorganisms B may gain entry into a fine pore not shown in the hydrophilic filter 7a. More specifically, part or all of a cell, though not shown, of the microorganism B may gain entry into an inside of the hydrophilic filter 7a.

In the filter separation method according to this embodiment, though not shown, a prescribed amount of an ATP eliminating reagent is dispensed in the first contact chamber 66, to thereby eliminate an ATP which is present outside a cell of the microorganism B. In this step, the ATP eliminating reagent passes through the hydrophilic filter 7a but is retained on the hydrophobic filter 7b due to the water repellency thereof, similarly to the above-described hot water HW. The second contact chamber 67 is thereby filled with the ATP eliminating reagent.

The above-described suction head 104a (see FIG. 4) sucks the ATP eliminating reagent in the first contact chamber 66 and the second contact chamber 67, to thereby discharge the ATP eliminating reagent out of the microorganism capturing device 1.

Examples of the ATP eliminating reagent include an ATP-degrading enzyme.

As shown in FIG. 5A4, a prescribed amount of the ATP extraction reagent EX is dispensed in the first contact chamber 66, to thereby extract an ATP (not shown in FIG. 5A4) present in the microorganism B (see FIG. 5B3).

Examples of the ATP extracting reagent EX include a benzalkonium chloride, a trichloroacetic acid, and a Tris buffer solution.

In this step, the ATP extraction reagent EX passes through the hydrophilic filter 7a but is retained on and above the hydrophobic filter 7b due to the water repellency thereof. The second contact chamber 67 is thereby filled with the ATP extraction reagent EX. As shown in FIG. 5B4, the ATP extraction reagent EX contains the ATP extracted from the microorganism B (see FIG. 5B3).

A prescribed amount of the ATP extraction reagent EX containing the ATP, that is, an ATP extract is dispensed and is put in a luminescence-test tube of a prescribed luminescence-intensity measurement unit. The ATP extract reacts with an ATP luminescence reagent in the luminescence-test tube and produces luminescence at a luminescence intensity corresponding to an ATP amount in proportion to the number of the microorganisms B.

Examples of the ATP luminescence reagent include a luciferase/luciferin reagent.

A luminescence detecting unit equipped with a photomultiplier tube measures the above-described luminescence intensity, to thereby obtain a relative number of the microorganisms B captured by the carrier 5 of the microorganism capturing device 1, based on the luminescence intensity.

More specifically, a luminescence detection signal which is detected and outputted by the luminescence detecting unit is digitally processed, to thereby measure a luminescence intensity (CPS) based on a single-photon counting method. An ATP amount (amol) contained in the ATP extract dispensed into the luminescence-test tube is calculated based on, for example, a prescribed map (calibration curve) indicating relation between ATP amounts (amol) and luminescence intensities (CPS). The number of the microorganisms is then counted by using an ATP value converted into the number of the microorganisms B captured in the carrier 5, based on the ATP amount (amol) and the prescribed amount of the dispensed ATP extract. Then, a series of steps of the method of counting the microorganisms B captured by the microorganism capturing device 1 according to this embodiment terminate.

Next are described advantageous effects of the microorganism capturing device 1 (filter separation apparatus) and the filter separation method according to this embodiment.

As described above, in the microorganism capturing device 1 and the filter separation method according to this embodiment, the hydrophilic filter 7a and the hydrophobic filter 7b are disposed spaced apart from each other. This allows a treatment agent such as the above-described reagent to pass through the hydrophilic filter 7a and to retain the treatment agent on the hydrophobic filter 7b due to the water repellency thereof. That is, the treatment agent such as the above-described reagent is present both in the first contact chamber 66 and in the second contact chamber 67 via the hydrophilic filter 7a.

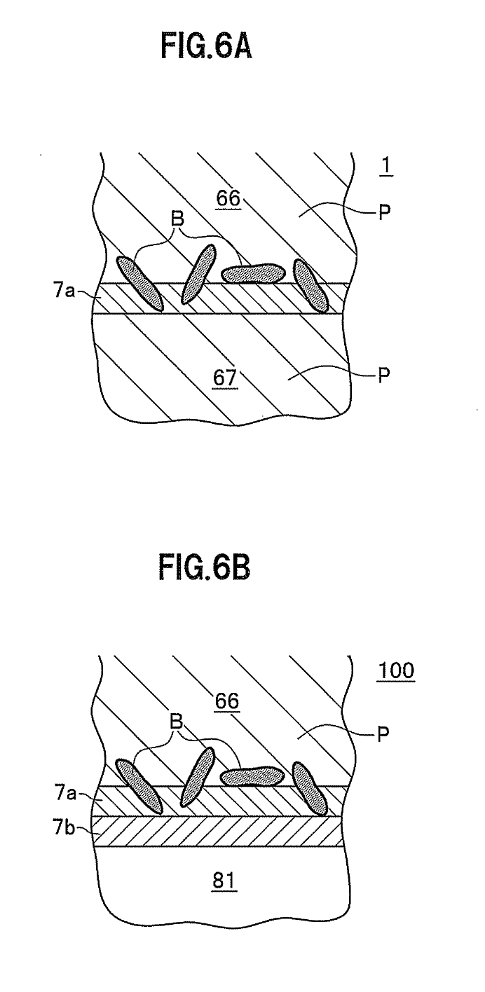

FIG. 6A to be referred to next is a schematic diagram illustrating a state in which, in the microorganism capturing device 1 (filter separation apparatus) of FIG. 1, microorganisms on the hydrophilic filter 7a are brought in contact with an ATP extraction reagent. FIG. 6B is a schematic diagram illustrating a state in which, in a microorganism capturing device (filter separation apparatus) according to a conventional technology, microorganisms on the hydrophilic filter 7a are brought in contact with an ATP extraction reagent. Note that the microorganism B shown in FIG. 6A and FIG. 6B have each a micrometer size (for example, a staphylococcus is approximately 1 μm long and an E. coli is approximately 0.5 μm in shorter diameter). Meanwhile, the hydrophilic filter 7a is, for example, approximately 35 μm thick, though the thickness varies depending on various commercially available products because of their different standards). FIG. 6A and FIG. 6B are thus not illustrated based on an actual relative scale.

As shown in FIG. 6B, in a microorganism capturing device 100 (see, for example, the above-described Japanese Laid-Open Patent Application, Publication No. 2012-053057), the hydrophilic filter 7a and the hydrophobic filter 7b are disposed directly one on top of the other. The microorganisms B are suction filtered to be separated and left on the hydrophilic filter 7a or the hydrophobic filter 7b. The filter separated microorganisms B are brought in contact with the treatment agent P, for example, a buffer solution, an ATP eliminating reagent, and an ATP extraction reagent.

In some cases, part or all of a cell, though not shown, of the filter separated microorganism B may gain entry into an inside of the hydrophilic filter 7a, as shown in FIG. 6B.

That is, in the microorganism capturing device 100 according to the conventional technology, when, for example, the treatment agent P is put in the microorganism capturing device 100, the microorganism B having been entered the hydrophilic filter 7a comes in contact not only with the treatment agent P which infiltrates the hydrophilic filter 7a but also with the treatment agent P which is retained mainly on an upper surface of the hydrophilic filter 7a.

In FIG. 6B, designated at the reference numeral 81 is the filtrate outlet.

On the other hand, in the microorganism capturing device 1 according to this embodiment, when the treatment agent P is put in the microorganism capturing device 1, as shown in FIG. 6A, both the first contact chamber 66 and the second contact chamber 67 which are positioned on both sides of the hydrophilic filter 7a are filled with the treatment agent P.

That is, the microorganism B entering into the hydrophilic filter 7a comes in contact with the treatment agent P from both the first contact chamber 66 and the second contact chamber 67.

Thus, in the microorganism capturing device 1 according to this embodiment, efficiency in contacting microorganism B with the treatment agent P is improved. As a result, a pretreatment (an operation of contacting an object to be treated with a treatment) can be performed effectively in counting the microorganisms B (object to be treated) counted in accordance with the ATP method.

Hence, the microorganism capturing device 1 (filter separation apparatus) and the filter separation method according to this embodiment make it possible to increase an ATP amount in an ATP extract, compared to the conventional microorganism capturing device 100 (see FIG. 6B). This allows sensitivity for counting the microorganisms B to be improved.

In this embodiment, examples of a treatment of the microorganisms B (object to be treated) with the treatment agent P include: a treatment of eliminating an ATP present out of a cell of the microorganism B using an ATP eliminating reagent (the treatment agent P); and a treatment of extracting an ATP present in the microorganism B using an ATP extraction reagent (treatment agent P).

In this embodiment, another treatment of the microorganism B (object to be treated) with the treatment agent P includes, as shown in FIG. 5B2, a treatment of diluting the microorganism B (object to be treated) contained in the carrier 5 with the hot water HW (as the treatment agent P). In the treatment, as shown in FIG. 5B2, the hot water HW is added to the carrier 5 containing the microorganism B of FIG. 5B1. When the hot water HW infiltrates the second contact chamber 67 via the hydrophilic filter 7a, though not shown, one or more of the microorganisms B may gain entry into the inside of the hydrophilic filter 7a.

At this time, the hot water HW in the second contact chamber 67 comes in contact with the microorganism B which enters into the hydrophilic filter 7a, also from the second contact chamber 67. The microorganism B to which a component of the carrier 5 is attached is brought in contact with the hot water HW from both the first contact chamber 66 and the second contact chamber 67. This further facilitates solation of the component of the carrier 5 attached to the microorganism B. This makes it easier for the component of the carrier 5 to be removed from the microorganism B in a subsequent suction filtration step.

The embodiment of the present invention has been explained as described above. The present invention is, however, not limited to the embodiment and can be carried out with various modifications.

The microorganism capturing device 1 (filter separation apparatus) and the filter separation method according to this embodiment each have a structure in which the hydrophilic filter 7a and the hydrophobic filter 7b are disposed apart by interposing the ring-shaped spacer 9 therebetween. However, the present invention is not limited to the structure having the ring-shaped spacer 9, as long as the hydrophilic filter 7a and the hydrophobic filter 7b are disposed with spacing from each other.

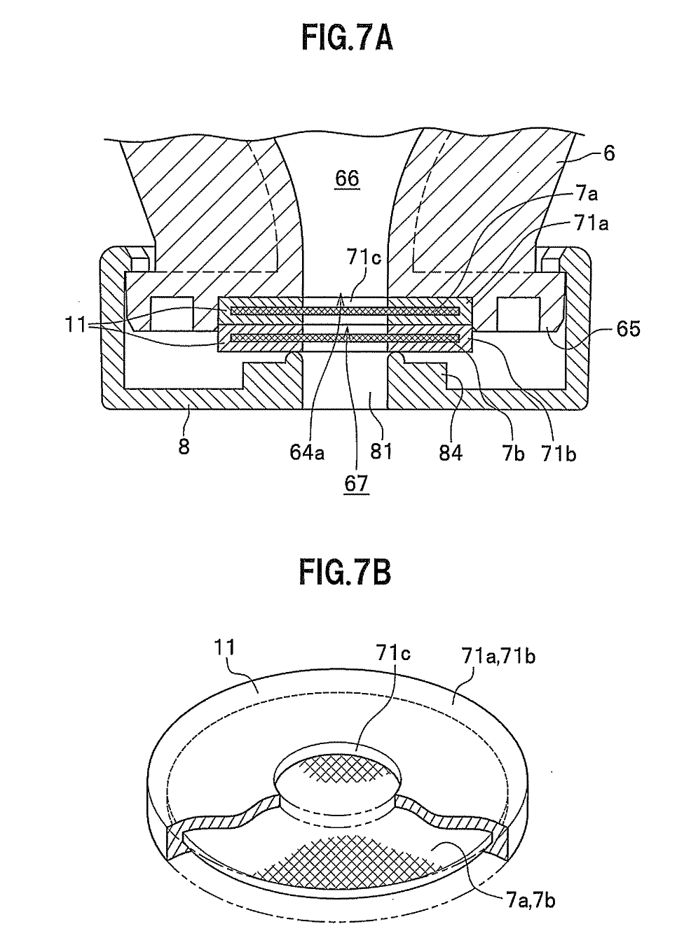

FIG. 7A to be referred to next is a partially enlarged cross sectional diagram illustrating a filter fitting portion and a vicinity thereof of a microorganism capturing device (filter separation apparatus) according to a variation of the present invention. FIG. 7B is a perspective diagram illustrating a filter hydrophilic filter forming body 71a and a hydrophobic filter forming body 71b used in the microorganism capturing device of FIG. 7A with a partial cutaway of a support member of the filters, according to the variation of the present invention.

As shown in FIG. 7A and FIG. 7B, the microorganism capturing device 1 according to the variation includes the filter hydrophilic filter forming body 71a and the hydrophobic filter forming body 71b, instead of an assembly constituted by the hydrophilic filter 7a (see FIG. 4), the hydrophobic filter 7b (see FIG. 4), and the spacer 9 (see FIG. 4) according to the above-described embodiment.

The hydrophilic filter forming body 71a includes: the hydrophilic filter 7a; and a substantially ring-shaped support member 11 formed along a circumferential edge of the hydrophilic filter 7a.

The hydrophobic filter forming body 71b includes: the hydrophobic filter 7b; and another substantially ring-shaped support member 11 formed along a circumferential edge of the hydrophobic filter 7b.

The hydrophilic and hydrophobic filter forming bodies 71a, 71b define a filtration part 71c which corresponds to a central hole portion surrounded by the substantially ring-shaped support members 11.

When the hydrophilic filter forming body 71a is aligned on top of the hydrophobic filter forming body 71b, the microorganism capturing device 1 has a structure in which the respective support members 11 of the hydrophilic filter forming body 71a and the hydrophobic filter forming body 71b come in contact with each other. This allows the hydrophilic filter 7a and the hydrophobic filter 7b to be disposed with spacing from each other. The support members 11 thus serve as a spacer.

In FIG. 7A, designated at the reference numeral 6 is the housing; at 8, the filter holding cap; at 64a, the discharge opening; at 65, the filter fitting portion; at 66, the first contact chamber; at 67, the second contact chamber; at 81, the filtrate outlet; and at 84, the seat portion.

The hydrophilic filter 7a and the hydrophobic filter 7b equipped with the respective support members 11 can be manufactured by insert molding with resin.

The resin used for the insert molding, that is, for forming the support members 11 is not specifically limited as long as the resin is thermoplastic. Elastomeric resin is preferably but not necessarily used because of its excellent performance of sealing between the filter fitting portion 65, the filter holding cap 8, the hydrophilic filter 7a, and the hydrophobic filter 7b.

Examples of the above-described elastomeric resin includes, for example: elastomer such as SBS, SIS, SEBS, and SEPS; an ethylene copolymer such as an ethylene-vinyl acetate copolymer and an ethylene ethyl acrylate copolymer; olefin resin such as atactic polypropylene, isotactic polypropylene, propylene-1-butene copolymer, and propylene-1-butene-ethylene copolymer; and ethylene-propylene rubber.

In the embodiment, the filtrate outlet 81 formed in the filter holding cap 8 is constituted by a single through hole. In the present invention, however, the filtrate outlet 81 may be constituted by a plurality of through holes (for example, honeycomb structural through holes).

In the embodiment, by using the microorganism counting apparatus 10, microorganisms captured by the microorganism capturing device 1 are counted. However, the present invention can also be realized by manually dispensing a reagent into the housing 6 and counting microorganisms using the ATP method, without mounting the microorganism capturing device 1 in the microorganism counting apparatus 10.

The present invention is applicable to a spore forming bacterium such as Bacillus subtilis. In this case, the above-described examples of the reagent can include a vegetative cell conversion reagent such as amino acid and sugar.

In the embodiment, by using the ATP method, microorganisms are counted. However, the present invention can also be realized by counting microorganisms based on fluorescence produced when an in-vivo substance such as a DNA, a RNA, and a NAD extracted from a microorganism is irradiated with excitation light.

In a case where the microorganism capturing device 1 is used for capturing and counting gram negative bacilli, the microorganisms can be counted as follows. Endotoxin contained in cell membranes of the microorganisms is set as a reference and the microorganisms are counted based on luminescence intensity obtained by making the endotoxin react with limulus.

The microorganism capturing device 1 of the present invention may capture microscopic particles of metal or of a chemical substance. The microorganism capturing device 1 may capture an object such as, but not limited to, a solid and mist.

1. A filter separation method for filter-separating an object to be treated, in which: the object to be treated is contacted with a liquid treatment agent; a first filter and a second filter are used; and the second filter exhibits liquid repellency to the liquid treatment agent, the filter separation method comprising the steps of:
disposing the first filter and the second filter spaced apart from each other; and
putting a mixture of the object to be treated and the treatment agent onto the first filter from a side opposite to the second filter, separating the object to be treated on the first filter, allowing the treatment agent in the mixture to pass through the first filter and to be retained between the first filter and the second filter due to the liquid repellency of the second filter, without passing through the second filter.

2. The filter separation method according to claim 1, a plurality of the treatment agents of different types being provided, the filter separation method further comprising the steps of:
forcibly discharging, through the second filter, a first treatment agent of the plurality of the treatment agents which fills between the first filter and the second filter, against the liquid repellency of the second filter; and
putting a second treatment agent of the plurality of the treatment agents onto the first filter from the side opposite to the second filter, allowing the second treatment agent to pass through the first filter and to be retained between the first filter and the second filter due to the liquid repellency of the second filter, without passing through the second filter.

3. The filter separation method according to claim 1,
wherein the first filter is a hydrophilic filter and the second filter is a hydrophobic filter.

4. A filter separation apparatus used for a filter separation method for filter-separating an object to be treated, comprising:
a first contact chamber and a second contact chamber in each of which the object to be treated is contacted with a treatment agent;
a filtrate outlet from which a filtrate made up of the object to be treated and the treatment agent is discharged outside;
a hydrophilic filter that separates the first contact chamber and the second contact chamber from each other; and
a hydrophobic filter that separates the second contact chamber and the filtrate outlet from each other.

5. The filter separation apparatus according to claim 4,
wherein the second contact chamber is formed by separating the hydrophilic filter and the hydrophobic filter from each other by a spacer.

6. The filter separation apparatus according to claim 5,
wherein the spacer is formed by a ring member, and the second contact chamber is formed inside the ring member.
